(19) United States
(12) Patent Application Publication
(10) Pub. No.: US 2016/0184147 A1
(43) Pub. Date: Jun. 30, 2016

(54) DEVICE FOR APPLYING AN INTERFACE DRESSING

(71) Applicant: URGO RECHERCHE INNOVATION ET DEVELOPPEMENT, Chenove (FR)

(72) Inventors: Anne-Sophie AMEY, Perrigny-Les-Dijon (FR); Nadège DESMAISON, Tart Le Haut (FR)

(73) Assignee: URGO RECHERCHE INNOVATION ET DEVELOPPEMENT, Chenvoe (FR)

(21) Appl. No.: 14/910,076

(22) PCT Filed: Jul. 30, 2014
(86) PCT No.: PCT/EP2014/066405

§ 371 Date: Feb. 4, 2016

(30) Foreign Application Priority Data
Aug. 5, 2013 (FR) 1357785

(51) Int. Cl. A61F 15/00; A61F 13/02; A61F 13/00

(52) U.S. Cl. CPC A61F 15/005; A61F 13/00085; A61F 13/0266

(57) ABSTRACT

The present invention relates to a device (20) for applying an interface dressing, including an interface dressing (22) which includes a first contact surface (24) and a second contact surface (28), the first contact surface (24) being suitable for being applied in contact with the skin or with a wound of a patient, a pealable application tab (32, 54) of the interface dressing (22), the application tab (32, 54) being arranged against the second contact surface (28), a portion (34, 6) for gripping the application tab (32), extending beyond the second contact surface (28), and a first pealable protector (26) and second pealable protector (30) arranged against the first contact surface (24) and against the second contact surface (28), respectively, the second protector (30) being pealable independently of the application tab (32). The invention also relates to a method for applying such an interface dressing on a patient.

The present invention relates to a device for applying an interface dressing comprising peelable protectors and at least one peelable application tab. The invention also relates to a method for applying such an interface dressing to a patient.

In the field of wound treatment it is known to use a dressing intended to be applied directly to a wound ensuring an interface between said wound and any absorbent compress arranged after the application of said dressing in order to absorb any exudates coming from the wound. Such a dressing is usually referred to by the term “interface dressing”. Among the known interface dressings, the product marketed by Laboratoires URGO under the name Urgotul® can be mentioned for example.

For the protection and application of interface dressings, an interface dressing is known from FR 2 783 412 A1 each of the contact surfaces of which is covered by a polyester film. A first polyester film can be removed from the first face in order to allow the application of the interface dressing from the side of the first face onto the patient's wound. After application of the interface dressing, the second polyester film is removed from the second face in order to allow the application of a compress or of a pad absorbing the exudates from the wound through the interface dressing. Such an application of the interface dressing to the skin is complicated if it is desired to avoid any contamination through contact of the fingers of the user with the interface dressing. This application of the interface dressing remains complicated even in the case where the user is wearing gloves because the interface dressing may adhere to the gloves.

Another device for protecting an interface dressing is known from WO 2008/145884 A1 consisting of a single polyester film covering the two faces of the interface dressing. The polyester film can be removed from the first face by means of a free portion in order to make it easier to apply the interface dressing to the skin or the wound of the patient. However, an optimal application of the dressing remains complicated because the polyester film totally covers the second face of the interface dressing. In effect, this limits the possibilities of adapting the interface dressing to the contours of the area to which it is to be applied. Furthermore, the application of the interface dressing after even partial removal of the polyester film from the second face does not allow all risk of contamination by the fingers of the user to be avoided.

If an interface dressing is more fragile and more flexible than a conventional dressing, i.e. a dressing with a single contact surface, an inappropriate application of an interface dressing may cause the interface dressing to be altered.

There is therefore a need for an improved interface dressing, the handling of which is simplified for the application of said interface dressing to the skin or to a wound of a patient, and that without altering said interface dressing.

More particularly, the invention aims to provide a device for applying an interface dressing making it easier to apply an interface dressing without altering it.

To this end, the present invention proposes a device for applying an interface dressing comprising:
an interface dressing comprising a first and a second contact surfaces, the first contact surface being suitable for being applied in contact with the skin or a wound of a patient;
a peelable application tab of the interface dressing, the application tab being arranged against the second contact surface, a gripping portion of the application tab extending beyond the second contact surface;
a first and a second peelable protectors arranged against the first and the second contact surfaces, respectively, the second protector being peelable independently of the application tab.

According to the preferred embodiments, the invention comprises one or more of the following characteristics, taken alone or in combination:
a first and a second protector which completely cover the first and the second contact surfaces, respectively;
a second protector which extends beyond the second contact surface by means of a gripping portion;
a second protector which overlaps the application tab;
preferably, a part of the first protector which forms a single piece with the application tab;
another peelable application tab with a gripping portion, the application tabs being arranged on two opposite sides of the second contact surface, the gripping portion of each of the application tabs extending beyond the second contact surface;
a first protector which has a peeling starter portion of said first protector; and
a first protector which is formed of two pieces each provided with a peeling starter portion folded onto the remainder of the first protector towards the exterior of the application device with respect to the interface dressing.

The invention also proposes a method for applying an interface dressing comprising the steps consisting of:
providing a device for applying an interface dressing as described previously;
removing the first protector from the first contact surface;
gripping the interface dressing by means of the application tab or, where appropriate, tabs;
applying the first contact surface to the skin or the wound of a patient using the application tab or tabs, the second protector being peeled off the second contact surface, either prior to or after the application of the first contact surface to the patient;
removing the application tab or tabs.

According to a preferred embodiment, the method comprises a step before the removal of the application tab or tabs and after the peeling off of the second protector and the application of the first contact surface to the skin or the wound of the patient, of the arrangement of a compress or of an absorbent pad against the second contact surface, the peeling off of the application tab or tabs being carried out while holding the interface dressing on the skin or the wound by means of the compress or the absorbent pad.

Other characteristics and advantages of the invention will become apparent on reading the description which follows of a preferred embodiment of the invention, given by way of example and with reference to the attached drawings.

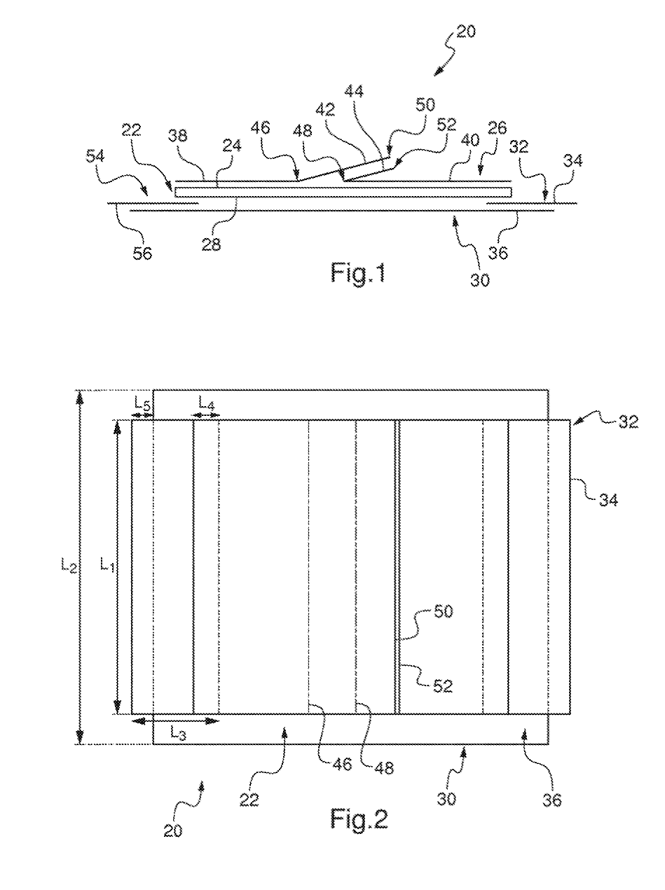

FIG. 1 represents a sectional view diagram of an interface dressing.

FIG. 2 represents a top view diagram of an interface dressing.

With reference to FIG. 1, a device 20 for applying an interface dressing is proposed comprising an interface dressing 22. The interface dressing 22 comprises a first face 24 suitable for application to the skin or a wound of a patient. Said first face 24 may also comprise, in an advantageous variant, a layer which can be solubilized when it comes into contact with the exudates coming from the wound. This solubilizable layer can comprise, in particular, an active ingredient chosen from the list constituted by but not limited to antibacterial agents, antiseptics, pain killers, anaesthetics, anti-inflammatory drugs, vitamins or even the oligosaccharides, or salts or complexes thereof. The interface dressing 22 also comprises a second face 28 opposite the first face 24. The second face 28 can be intended to receive a compress or a pad absorbing the exudates from the wound through the interface dressing 22.

In order to make handling prior to the application of the interface dressing 22 easier, the proposed device 20 for applying an interface dressing comprises a first and a second protectors, 26 and 30. These first and second protectors, 26 and 30, are respectively arranged against the first contact surface 24 and the second contact surface 28. In order to limit contamination prior to handling, the first and second protectors, 26 and 30, preferably cover at least completely the first and the second contact surfaces 24 and 28, respectively.

In order to allow the interface dressing 22 to be applied to the patient's wound, or optionally between a wound and a compress or an absorbent pad, the first and second protectors 26 and 30 are peelable. The term peelable is understood to mean that a support, such as the first or the second protector 26 or 30, can be detached from the interface dressing 22 to which it adheres without tearing either the support or the interface dressing 22. This removal, also called “peeling”, can in particular be carried out by pulling the support rather than by shearing, which makes it possible to limit the risk of damaging the interface dressing 22. Peeling the protector 26 or 30 off the interface dressing 22 can be carried out by pulling the protector 26 or 30 and the interface dressing 22 in two opposite directions along a direction perpendicular to the contact surfaces 24 and 28 of the interface dressing 22.

If the first face 24 of the interface dressing 22 is intended to be applied in contact with the skin or a wound of the patient, it is anticipated that the first protector 26 will be peeled from the first face 24 before the first face 24 is applied to the skin or the wound.

In order to make handling and application of the interface dressing 22 easier after the first protector 26 has been peeled off, the device for applying an interface dressing 20 also comprises an application tab 32 of the interface dressing 22 arranged against the second contact surface 28. This application tab 32 comprises a portion 34 extending beyond the second face 28. By extending beyond the second face 28, this portion 34 makes gripping possible without direct contact with the interface dressing 22. The absence of contact between the fingers of the user and the interface dressing 22 makes it possible not to contaminate the interface dressing 22 and to avoid any adhesion between the fingers or the gloves of the user and the interface dressing 22 without complicating the application of the interface dressing 22.

According to the preferred embodiment shown in FIG. 1, the interface dressing device comprises another application tab 54 arranged on the side opposite the application tab 32. This other application tab 54 also comprises a gripping portion 56 extending beyond the second contact surface 28 in order to make it easier to grip the interface dressing 22 without direct contact. This second tab 54 allows even easier handling and application of the interface dressing 22. In fact, the presence of the one or two application tabs 32 and 54 makes it possible to simplify handling of the interface dressing 22, such as for example when the latter is arranged so that it covers itself at least partially, but also to arrange it so that it fits optimally on a non-planar surface.

In order to make handling and application of the interface dressing 22 easier, the tabs 32 and 54 preferably only partially cover the second contact surface 28. In other words, a portion of the second contact surface 28 is preferably free from contact with the one or the other of tabs 32 and 54 and is protected from the external environment by the second protector 30. An only partial coverage of the second contact surface 28 of the interface dressing 22 by the tab or tabs 32 and 54 makes it possible to make handling and application of the interface dressing 22 to the skin or the wound of the patient easier. In fact, in this case, the interface dressing 22 is easier to use than in the case of almost total, or even total, coverage of the second contact surface 28 by the tab or tabs 32 and 54, in particular by making it possible for the interface dressing 22 to be folded on itself.

In addition, these tabs 32 and 54 can have bevelled shapes in particular at the angles of the part of the tabs 32 and 54 which are in contact with the second contact surface 28 of the interface dressing 22. These bevels make it possible to remove the tabs 32 and 54 more easily once the interface dressing 22 is arranged in contact with the skin or the wound of the patient.

The tabs 32 and 54 are peelable in order to allow their removal from the interface dressing 22 without altering the interface dressing 22 once it is applied to the skin or the wound. Nevertheless, the second protector 30 is peelable independently of the application tab or tabs 32 and 54. The second protector 30 can thus be removed from the second face 28 while leaving the application tab or tabs 32 and 54 on the second face 28. According to the embodiment shown in FIG. 1, the second protector 30 can be removed independently of the application tabs 32 and 54 by being arranged on the second contact surface 28 but above the application tabs 32 and 54, i.e. overlapping the application tabs 32 and 54. Alternatively, according to an embodiment which is not shown, the second protector 30 can be peeled off independently of the application tabs 32 and 54 by being arranged next to and at the same level as the application tabs 32 and 54 on the second contact surface 28. In this case, in order to limit contamination, it is preferred that no area of the second contact surface 28 is left uncovered between the second protector and the application tabs 32 and 54. In other words, it is preferred that the application tabs 32 and 54 and the second protector 30, all being independent, ensure complete coverage of the second contact surface 28. In precisely this case, it can be envisaged to add a starter portion to the protector 30 in order to make the removal of the second protector 30 easier and thus to limit the risk of contamination or of contact with fingers or gloves, in particular during its removal. This starter portion is preferably located on one of the edges of the protector 30.

A method is proposed for applying an interface dressing 22 using the above device for applying an interface dressing 20. When a user wishes to apply the interface dressing 22 of the device 20 for applying an interface dressing to the skin or a wound of a patient, he may proceed in a preferred manner according to the following steps: peeling off the first peelable protector 26; gripping the device 20 for applying an interface dressing by the gripping portion or portions 34 and 56 of the application tab or tabs 32 and 54; peeling off the second peelable protector 30; applying the first face 24 of the interface dressing 22 to the skin or wound and peeling off the application tab or tabs 32 or 54. Alternatively, the second protector 30 may be peeled off after the first contact surface 24 of the interface dressing 22 has been applied to the skin or the wound. The second protector 30 is then peeled off while holding the gripping portion or portions 34 and 56 of the application tab or tabs 32 and 54 on the skin and by gripping the second protector 30. However, it is preferred to remove the protector 30 before applying the interface dressing 22 to the skin or the wound of the patient. In fact, removing the second protector 30 makes it possible to make the interface dressing 22 easier to use than when the interface dressing 22 is provided with the second protector 30, in particular by making it easier to fold the interface dressing 22 on itself. In all cases, after the second protector 30 has been peeled off and the interface dressing 22 has been applied against the skin or the wound of the patient, a compress or absorbent pad can be arranged against the second contact surface 28. The tab or tabs 32 and 54 are preferably arranged around the periphery of the second contact surface 28, i.e. on the sides of the second contact surface 28 in order to leave free the major part of the second contact surface 28 which comes into contact with the compress or the absorbent pad. After the compress or the absorbent pad has been arranged on the second contact surface 28 between the tabs, preferably in an area of the second face 28 not covered by the tabs 32 and 54, the tabs 32 and 54 can be easily peeled off by pressing down, for example, on the compress or the absorbent pad in order to separate the application tab or tabs 32 and 54 from the interface dressing 22.

In other words, in the proposed method, the independence of the peeling off of the second protector 30 and the peeling off of the application tab or tabs 32 and 54 makes it possible for the user to have a bearing point on the second face 28 in order to make it easier to peel off the second protector 30. In addition, when the second protector 30 is peeled off, the application tab or tabs 32 and 54 make it possible to handle the interface dressing 22 without direct contact with the contact surfaces 24 and 28 of the interface dressing 22.

In order to make it easier to peel off the second protector 30, a gripping portion 36 of the second protector 30 can extend beyond the second surface 28. By extending beyond the second face 28, this portion 36 makes it possible to grip the second protector 30 in order to peel off the second protector 30 by separating the application tab 32 and the gripping portion 36 in two opposite directions. The peeling off of the second protector 30 is thus made easier.

Arranging the application tab or tabs 32 and 54 on the second face 28 is advantageous compared to an application tab arranged on the first face 24. Such a tab arranged on the face 24 of the dressing in contact with the skin or the wound is still arranged between this first face 24 and the skin or the wound after the dressing has been applied. This arrangement of the tab on the face of the dressing in contact with the skin then forces the patient user to remove the tab by shearing of the interface dressing 22 by pulling the gripping portion of the tab in a direction substantially parallel to the faces 24 and 28. However, if such an arrangement of the tab between the skin and the dressing can be envisaged for conventional dressings with a single contact surface as shown in FIG. 12 of document EP 0 161 865 A2, in such a case, there is the risk of damage to the more fragile and more flexible interface dressing 22. In fact, because of the fragility of the interface dressing 22, such a shearing can damage the interface dressing 22. In particular, the shearing of the interface dressing 22 can cause distortion or temporary or permanent changes, such as a rupture, splitting or creep of the interface dressing 22 which limits the effectiveness of the interface dressing 22. The proposed application device 20 with the tab or tabs 32 and 54 on the second contact surface 28 makes it possible to remove the tabs 32 and 54 by peeling, i.e. by pulling the gripping portions 34 and 54 in order to move them away from the second face 28 in a direction perpendicular to the second face 28. Such a removal by peeling of one of the tabs 32 and 54 ensures the integrity of the interface dressing 22 in comparison with removal by shearing.

Moreover, the arrangement of the application tab on the second face 28 has the advantage of a less painful removal for the patient compared to an arrangement of the tab between the interface dressing 22 and the patient. In fact, when the application tab is next to the patient, the tab, during its removal, risks rubbing the skin of the patient in an area near to the wound, i.e. the peri-lesional skin, which can be delicate and sensitive.

In addition, the removal by peeling permitted by the arrangement of the tabs 32 and 54 on the second contact surface 28 is much easier than removal by shearing when the dressing is applied on a less accessible area such as a concave or cavitary area.

The proposed application device 20 also has the advantage of allowing the complete removal of the application tab or tabs 32 and 54 from the dressing 22. In other words, because the tabs 32 and 54 are peelable, after application, the interface dressing 22 can be left on the skin without any other remnant from the proposed application device 20. This complete removal is advantageous compared to an application tab a portion of which remains arranged on the dressing such as for conventional dressing tabs, shown in FIGS. 5 and 6 of document EP 0 161 865 A2, which are either adherent and fixed to the skin around the dressing, or are partially removed. In fact, in the case of the interface dressing 22, the complete removal of the tab or tabs 32 and 54 ensures that there is no obstacle between the second contact surface 28 and the compress or the pad, i.e. all of the surface of the interface dressing 22 is used for the passage of the exudate from the wound through the interface dressing 22. In contrast, arranging tabs which remain at least partially fixed to the second contact surface, either because the tab fixes directly to the skin or because of the remains of a torn tab, constitutes an obstacle to the passage of the exudate from the wound through the interface dressing 22. Such an obstacle restricts at the least the passage of the exudate but also the absorption of said exudate by the compress or the absorbent pad, impairing the effectiveness sought for an interface dressing.

According to a variant shown in FIG. 1, the first protector 26 is formed of two pieces 38 and 40 each equipped with a peeling starter portion 42 or 44 of the first protector 26. The starter portions 42 and 44 are folded towards the exterior of the application device 20 against the interface dressing 22, in order to be accessible to the user. In particular, the starter portions 42 and 44 extend from folds 46 and 48 to ends 50 and 52, respectively. The starter portions 42 and 44 can be folded from the same side of the first protector 26, as shown, such that the starter portion 42 overlaps the starter portion 44. The overlapping of the starter portions 42 and 44 makes it possible that no area of the first contact surface is left uncovered between the two pieces 38 and 40, reducing contamination of the interface dressing 22. Alternatively, the starter portions 42 and 44 can be folded towards two opposite sides of the first protector 26, i.e. the starter portions 42 and 44 are folded towards the pieces 38 and 40, respectively, rather than towards one of the pieces 38 and 40. In order to limit contamination it is preferred, particularly in this latter case, for the folds 46 and 48 to be more or less identical.

According to another embodiment which is not shown, a part of the first protector 26 forms a single piece with an application tab 32 or 54. In the case where the first protector 26 is formed of the two pieces 38 and 40, each of the two pieces 38 and 40 of the first protector 26 can be formed in a single piece with one of the application tabs 32 and 54. In particular, a portion of each piece 38 and 40 can extend beyond the first face 24 in order to rejoin the application tab of the same side, 54 and 32, respectively. In this case, when the piece 38 is peeled off, it is still held at the second face 28 of the interface dressing 22 via the application tab 54. In a similar way, when the piece 40 is peeled off, it is still held at the second face 28 of the interface dressing 22 via the application tab 32. The absence of total coverage of the tabs 32 or 54 of the face 28 of the interface dressing 22 makes it easier to handle the interface dressing 22, both during gripping and in the application thereof to the skin or the wound of the patient.

The first and second protectors 26 and 30 and the tabs 32 and 54 are preferably made of polyolefin, preferably polyester, and preferably have a thickness of between 30 and 100 μm, preferably a thickness of between 40 and 80 μm and even more preferably a thickness of 50 μm. The interface dressing 22 can have a thickness of between 200 μm and 1 mm, preferably a thickness of between 400 and 600 μm and even more preferably a thickness of 500 μm.

According to the embodiment shown in FIGS. 1 and 2, the interface dressing 22 and the second protector 30 correspond respectively to a square of side L1 of 10 cm and a square of side L2 of 13 cm. According to this same embodiment, the application tabs 32 and 54 correspond to a rectangle with a width L3 of 3 cm and a length L1 of 10 cm. In addition, the contact areas between the application tabs 32 and 54 and the second face 28 correspond to a rectangle with a length L1 of 10 cm and a width L4 of 1 cm, the application tabs 32 and 54 exceeding the portion 36 of the second protector 30 by a length L5 of 3 cm.

Of course, the present invention is not limited to the examples and the embodiment described and shown but is capable of numerous variants available to a person skilled in the art. The first protector 26 can, in particular, be formed in a single piece covering the whole of the first contact surface 24 and have a single peeling starter portion extending beyond the first surface 24. Furthermore, the second protector 30 can be formed of two or more pieces covering the whole of the second contact surface 28 and the tab or tabs 32 and 54. Each of these pieces may have one or more starter portions.

1. Device for applying an interface dressing comprising:
an interface dressing comprising a first and a second contact surfaces, the first contact surface being suitable for being applied in contact with the skin or a wound of a patient;
a peelable application tab of the interface dressing, the application tab being arranged against the second contact surface, a gripping portion of the application tab extending beyond the second contact surface;
a first and a second peelable protectors arranged against the first and the second contact surfaces, respectively, the second protector being peelable independently of the application tab.

2. Application device according to claim 1 in which the first and the second protectors completely cover the first and the second contact surfaces, respectively.

3. Application device according to claim 1 in which the second protector extends beyond the second contact surface by means of a gripping portion.

4. Application device according to claim 1 in which the second protector overlaps the application tab.

5. Application device according to claim 1 in which a part of the first protector forms a single piece with the application tab.

6. Application device according to claim 1 comprising another peelable application tab with a gripping portion, the application tabs being arranged on two opposite sides of the second contact surface, the gripping portion of each of the application tabs extending beyond the second contact surface.

7. Application device according to claim 1 in which the first protector has a peeling starter portion of said first protector.

8. Application device according to claim 7 in which the first protector is formed of two pieces each equipped with a peeling starter portion folded onto the remainder of the first protector towards the exterior of the device against the interface dressing.

9. Method for applying an interface dressing comprising the steps consisting of:
providing a device for applying an interface dressing comprising:
an interface dressing comprising a first and a second contact surfaces, the first contact surface being suitable for being applied in contact with the skin or a wound of a patient
a peelable application tab of the interface dressing, the application tab being arranged against the second contact surface, a gripping portion of the application tab extending beyond the second contact surface;
a first and a second peelable protectors arranged against the first and the second contact surfaces, respectively, the second protector being peelable independently of the application tab;
removing the first protector from the first contact surface;
gripping the interface dressing by means of the application tab or, where appropriate, tabs;
applying the first contact surface to the skin or the wound of a patient using the application tab or tabs, the second protector being peeled off the second contact surface, either prior to or after the application of the first contact surface to the patient;
removing the application tab or tabs.

10. Method according to claim 9 comprising, before the removal of the application tab or tabs and after the peeling off of the second protector and the application of the first contact surface to the skin or the wound of the patient, the arrangement of a compress or of an absorbent pad against the second contact surface, the peeling off of the application tab or tabs being carried out while holding the interface dressing on the skin or the wound by means of the compress or the absorbent pad.

11. Method according to claim 9 in which the first and the second protectors completely cover the first and the second contact surfaces, respectively.

12. Method according to claim 9 in which the second protector extends beyond the second contact surface by means of a gripping portion.

13. Method according to claim 9 in which the second protector overlaps the application tab.

14. Method according to claim 9 in which a part of the first protector forms a single piece with the application tab.

15. Method according to claim 9 comprising another peelable application tab with a gripping portion, the application tabs being arranged on two opposite sides of the second contact surface, the gripping portion of each of the application tabs extending beyond the second contact surface.

16. Method according to claim 9 in which the first protector has a peeling starter portion of said first protector.

17. Method according to claim 16 in which the first protector is formed of two pieces each equipped with a peeling starter portion folded onto the remainder of the first protector towards the exterior of the device against the interface dressing.
